(19) United States
(12) Patent Application Publication
(10) Pub. No.: US 2025/0195161 A1
(43) Pub. Date: Jun. 19, 2025

(54) METHOD FOR ROBOTIC OPTICAL NAVIGATIONAL SURGERY SYSTEM

(71) Applicant: U.S. Patent Innovations, LLC, Takoma Park, MD (US)

(72) Inventors: Jerome Canady, Lakeland, FL (US); Cheffren Canady, Erie, CO (US); Taisen Zhuang, Rockeville, MD (US); Daniel Tabatabai, Annandale, VA (US); Tse Huai Wu, Fairfax, VA (US); Shruti Wigh, Silver Spring, MD (US); David Hinds, Silver Spring, MD (US)

(21) Appl. No.: 19/067,356

(22) Filed: Feb. 28, 2025

Related U.S. Application Data
(63) Continuation of application No. 16/759,636, filed on Apr. 27, 2020, now abandoned.
(60) Provisional application No. 62/609,042, filed on Dec. 21, 2017.

(51) Int. Cl. A61B 34/30; A61B 18/04; A61B 34/10; A61B 34/20

(52) U.S. Cl. CPC A61B 34/30; A61B 18/042; A61B 2034/107; A61B 2034/2057; A61B 2034/2065

(57) ABSTRACT

An automated robotic navigational surgical system that will detect dye (which is injected external to this system) that marks the areas of operation. The color and type of dye used will be one that is both distinct and highly reflective. There are four sections to the automated robotic navigational surgical system: Energy Source, Display Unit and Control Arm, Sensor Array, Disposable Tip.

### CROSS-REFERENCE TO RELATED APPLICATIONS

The present application is a continuation of U.S. application Ser. No. 16/759,636 filed on Apr. 27, 2020, which is a national stage of Application No. PCT/US2018/067072 filed on Dec. 21, 2018, which claims the benefit of the filing date of U.S. Provisional Patent Application Ser. No. 62/609,042 filed by the present inventors on Dec. 17, 2017.

The aforementioned patent applications are hereby incorporated by reference in their entirety.

### STATEMENT REGARDING FEDERALLY SPONSORED RESEARCH OR DEVELOPMENT

None.

### BACKGROUND OF THE INVENTION

### Field of the Invention

The present invention relates to robotic surgical systems, and more specifically to a navigation system for a robotic surgical system.

### Brief Description of the Related Art

A variety of minimally invasive robotic (or “telesurgical”) systems have been developed to increase surgical dexterity as well as to permit a surgeon to operate on a patient in an intuitive manner. Many of such systems are disclosed in the following U.S. patents which are each herein incorporated by reference in their respective entirety: U.S. Pat. No. 9,408,606, entitled “Robotically powered surgical device with manually-actuatable reversing system,” U.S. Pat. No. 5,792,135, entitled “Articulated Surgical Instrument For Performing Minimally Invasive Surgery With Enhanced Dexterity and Sensitivity”, U.S. Pat. No. 6,231,565, entitled “Robotic Arm DLUS For Performing Surgical Tasks”, U.S. Pat. No. 6,783,524, entitled “Robotic Surgical Tool With Ultrasound Cauterizing and Cutting Instrument”, U.S. Pat. No. 6,364,888, entitled “Alignment of Master and Slave In a Minimally Invasive Surgical Apparatus”, U.S. Pat. No. 7,524,320, entitled “Mechanical Actuator Interface System For Robotic Surgical Tools”, U.S. Pat. No. 7,691,098, entitled Platform Link Wrist Mechanism”, U.S. Pat. No. 7,806,891, entitled “Repositioning and Reorientation of Master/Slave Relationship in Minimally Invasive Telesurgery”, and U.S. Pat. No. 7,824,401, entitled “Surgical Tool With Wristed Monopolar Electrosurgical End Effectors.”

Recently a new treatment field called “Cold Atmospheric Plasma” has developed treating and/or removing cancerous tumors while preserving normal cells. For example, Cold Atmospheric Plasma systems, tools and related therapies have been disclosed in WO 2012/167089 entitled “System and Method for Cold Plasma Therapy,” US-2016-0095644-A1 entitled “Cold Plasma Scalpel,” US2017-0183632-A1 entitled “System and Method for Cold Atmospheric Plasma Treatment on Cancer Stem Cells,” and US-2017-0183631-A1 entitled “Method for Making and Using Cold Atmospheric Plasma Stimulated Media for Cancer Treatment.” The foregoing published patent applications are hereby incorporated by reference in their entirety. With such treatment cancerous tumor removal surgery can remove macroscopic disease that has been detected but some microscopic foci might remain.

Additionally, advances have been made in fluorescence guided surgery. In such systems, data visualization provides a step between signal capture and display needed for clinical decisions informed by that signal. For example, J. Elliott, et al., “Review of fluorescence guided surgery visualization and overlay techniques,” BIOMEDICAL OPTICS EXPRESS 3765 (2015), outlines five practical suggestions for display orientation, color map, transparency/alpha function, dynamic range compression and color perception check. Another example of a discussion of fluorescence-guided surgery is K. Tipirneni, et al., “Oncologic Procedures Amenable to Fluorescence-guided Surgery,” Annals of Surgery, Vo. 266, No. 1, July 2017).

### SUMMARY OF THE INVENTION

Identifying optical screening methods to locate tumors within biological tissue remains a challenge. Smart beacons targeting cancer tumors are being developed at an increasingly rapid pace. Bio-Imaging techniques in combination with surgery have improved because of the identification of over expressed biomarkers-receptors in cancerous tissues which are down-regulated in normal tissue. The primary goal in treating patients with cancer is to detect the cancer, complete resection of the tumor and to determine margins of the resected tissue are cancer free.

Optical smart beacons such as; green fluorescent protein (GFP), red fluorescent protein (RFP), metallic (i.e. gold) nanoparticles, semiconductor quantum dots (QDs), molecular beacons, and fluorescent dyes have been developed to identify over-expressed receptors on cancer cells and subsequently attached on the cells resulting in a fluorescent light beacon. These imaging techniques allow the surgeon, investigator to observe in real time the function of the cancer in humans or animals which include i.e. cell cycle position, apoptosis, metastasis, mitosis, invasion and angiogenesis. The cancer cells and supportive tissue can be color-coded which allows real time macro and micro-imaging technologies. A new field In Vivo Cell Biology has arisen.

We can currently identify cancerous tumors at the microscopic (applying microscopy) and macroscopic 2D and 3D applications by using optical imaging guided techniques. The ability of a Robotic Optical Navigational System (RONS) to robotically detect Bio Optic Image of cancerous tissue, process this images, map out and locate the image, transfer the image to 3D mapping coordinates and subsequently send the data to an energy source then deliver an energy beam (i.e. plasma) or electrical charge to exact mapped out location within the animal or human previously did not exist. A fully Robotic Optical Navigational System will be integrated optical imaging, navigational and deliver a plasma beam, or electrical charge to ablate or kill the tumor or any identify biological tissue which requires ablation.

The present invention provides a novel innovation for precise and uniform application of Cold Atmospheric Plasma using an automated robotic arm driven by preoperative CT, MRI or Ultrasound image guidance and/or fully automated robotic navigation using fluorescent contrast agents for a fluorescence-guided procedure. Dosage parameters may be set based on the type of cancer being addressed and stored genomic plasma results. The present invention further provides precise automated and uniform dosage of cold plasma for cancer treatment and wound care and precise automated control of a robotic surgical arm for other applications.

In a preferred embodiment, the present invention is an automated robotic navigational surgical system that will detect dye (which is injected external to this system) that marks the areas of operation. The color and type of dye used will be one that is both distinct and highly reflective. There are four sections to the automated robotic navigational surgical system: Energy Source, Display Unit and Control Arm, Sensor Array, Disposable Tip.

In another preferred embodiment, the present invention is a method for performing automated robotic surgical treatments. The method comprises scanning a patient for cancerous tissue in a plurality of regions in said patient, storing in a memory images of first and second regions of cancerous in said patient, analyzing cancerous tissue in each of said first and second regions of cancerous tissue to identify a type of cancerous tissue in each of the first and second regions of cancerous tissue, determining first specific cold atmospheric plasma dosage and treatment settings for cancerous tissue in said first region of cancerous tissue, determining second specific cold atmospheric plasma dosage and treatment settings for cancerous tissue in said second region of cancerous tissue, programming a robotic surgical system to move to the first region of cancerous tissue, locate cancerous tissue in that region, and apply cold atmospheric plasma of said first specific dosage and treatment settings to the first cancerous tissue, after completion of treatment of the first region move to the second region, locate the cancerous tissue in the second region and apply cold atmospheric plasma to that second cancerous tissue of the second specific dosage and settings. Further, robotic surgical system may locate cancerous tissue in a region by comparing stored images of said region to real-time images of said region.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a diagram illustrating the architecture of a system in accordance with a preferred embodiment of the present invention.

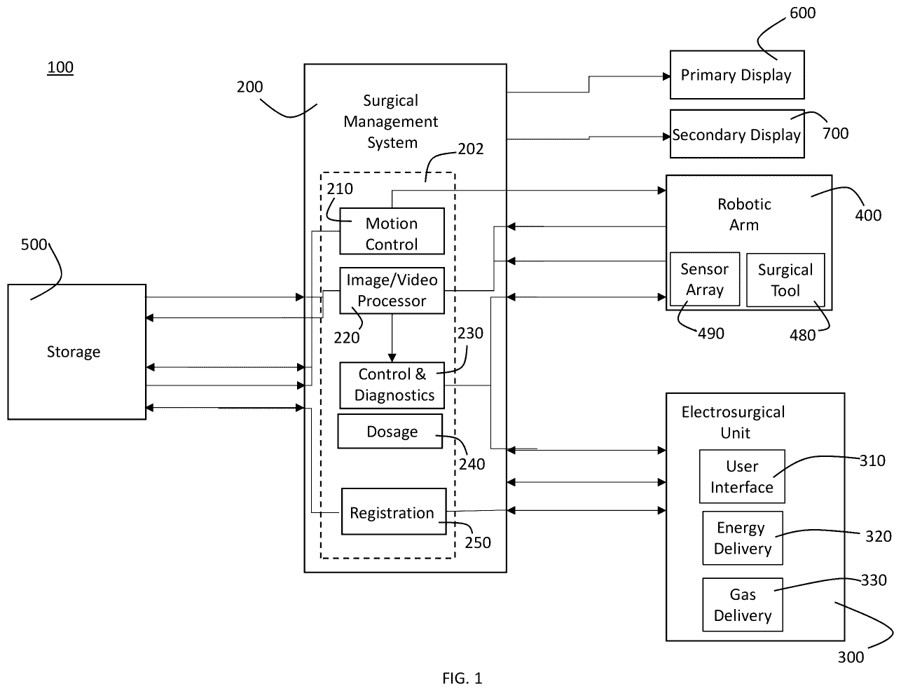

FIG. 2 is a diagram of a robotic surgical system in accordance with a preferred embodiment of the present invention.

FIG. 3 is diagram illustrating use of an optical smart beacon or dye to mark cancerous tissue.

FIG. 4 is diagram illustrating operation of a robotic surgical navigation system in accordance with a preferred embodiment of the present invention to locate cancerous tissue and sequence an energy beam to ablate or kill the cancerous tissue.

### DETAILED DESCRIPTION OF THE PREFERRED EMBODIMENTS

The preferred embodiments of the inventions are described with reference to the drawings.

In a preferred embodiment, a robotic navigation system 100 in accordance with the present invention has a surgical management system 200, an electrosurgical unit 300, a robotic control arm 400, a storage 500, a primary display 600 and a secondary display 700. A disposable tip or tool 480 and a sensor array or camera unit 490 are mounted on or incorporated into the robotic control arm 400. The electrosurgical unit 300 provides for a variety of types of electrosurgery, including cold atmospheric plasma, argon plasma coagulation, hybrid plasma cut, and other conventional types of electrosurgery. As such, the electrosurgical unit provides both electrical energy and gas flow to support the various types of electrosurgery. The electrosurgical unit preferably is a combination unit that controls deliver of both electrical energy and gas flow, but alternatively may a plurality of units such that one unit controls the electrical energy and another unit controls the flow of gas.

The surgical management system 200 provides control and coordination of the various subsystems. The surgical management system 200 has processors and memory 202 for storing and running software to control the system and perform various functions. The surgical management system has a motion control module or modules 210 for controlling movement of the robotic arm 400, an image/video processor 220, a control and diagnostics modules 230, a dosage module 240 and a registration module 250. The surgical management system 200 and the electrosurgical unit 300 may form an integrated unit, for example, such as is disclosed in International Application No. PCT/US2018/026894, entitled “GAS-ENHANCED ELECTROSURGICAL GENERATOR.”

The system electronic storage 500, which may be a hard drive, solid state memory or other known memory or storage, stores patient information collected in advance of and during surgical procedures. Patient information such as digital imaging may be 2D or 3D and may be performed via CT Scan, MRI, or other know methods to identify and/or map a region of interest (ROI) in a patient's body. In this way an area or areas of interest can be identified. These mapped images are uploaded from the storage 500 to the surgical management system 200 and interlaced with the current imagery provided by the onboard visual and IR cameras in the sensor array 490. Additionally, this imagery will allow the user to define target areas prior to scanning to increase the reliability of all subsequent scans and provide better situational awareness during the procedure. Preoperative planning and review may be performed using 2D/3D dataset in storage 500 to identify a target region or regions of interest in the patient. Preoperative information may include, for example, information regarding location and type of cancerous tissue and appropriate dosage or treatment settings information for the type of cancerous tissue to be treated. The type of cancerous tissue may be determined, for example, through biopsy and testing performed in advance of surgery. The dosage or treatment settings information may be retrieved from tables previously stored in memory or may be determined through advance testing on the cancerous tissue obtained via biopsy.

The preoperative patient information further can be used to program the surgical management system to perform a procedure. As an example, consider a patient for which the preoperative scanning an evaluation finds two regions having cancerous tissue and identifies the type of cancerous tissue in each region. The surgical management system can be programmed to seek out the first region of cancerous tissue, locate the cancerous tissue in that region, and apply cold atmospheric plasma of a specific dosage or treatment settings to that first cancerous tissue. After completion of treatment of the first region, the surgical management system moves the robotic arm to the second region, where is locates the cancerous tissue and applies cold atmospheric plasma to that second cancerous tissue of a dosage that is specific to that second cancerous tissue. In the context of cold atmospheric plasma, the “dosage” may include application time, power setting, gas flow rate setting and waveform or type of treatment (in this instance Cold Atmospheric Plasma).

During a procedure, visible light images and video may be shown on the primary display 600 and/or the secondary display 700. Images, video and metadata collected during a procedure by the sensor array 490 are transmitted to the surgical management system 200 and stored in the storage 500.

The advanced robotic arm 400 and camera unit 490 provide a compact and portable platform to detect target tissue such as cancer cells through guided imagery such as fluorescent navigation with the end goal being, for example, to administer cold plasma or other treatments to the target tissue. While examples are shown where the target tissue is cancerous tissue, other types of procedures such as knee replacement surgery can be performed using a robotic optical navigation system in accordance with the present invention. The plasma application will be a significant improvement from hand applied treatments. The surgical application of treatments such as cold plasma will be precise with respect to region of interest coverage and dosage. If necessary, the application can be repeated precisely. The sensor array 490 may comprise, but is not limited to, video and/or image cameras, near-infrared imaging to illuminate cancer cells, and/or laser/LIDAR for contour mapping and range finding of the surgical area of the patient. HD video and image acquisition from the sensor array 490 will provide the operator with an unprecedented view of the cold plasma application and provide reference recordings for future viewing.

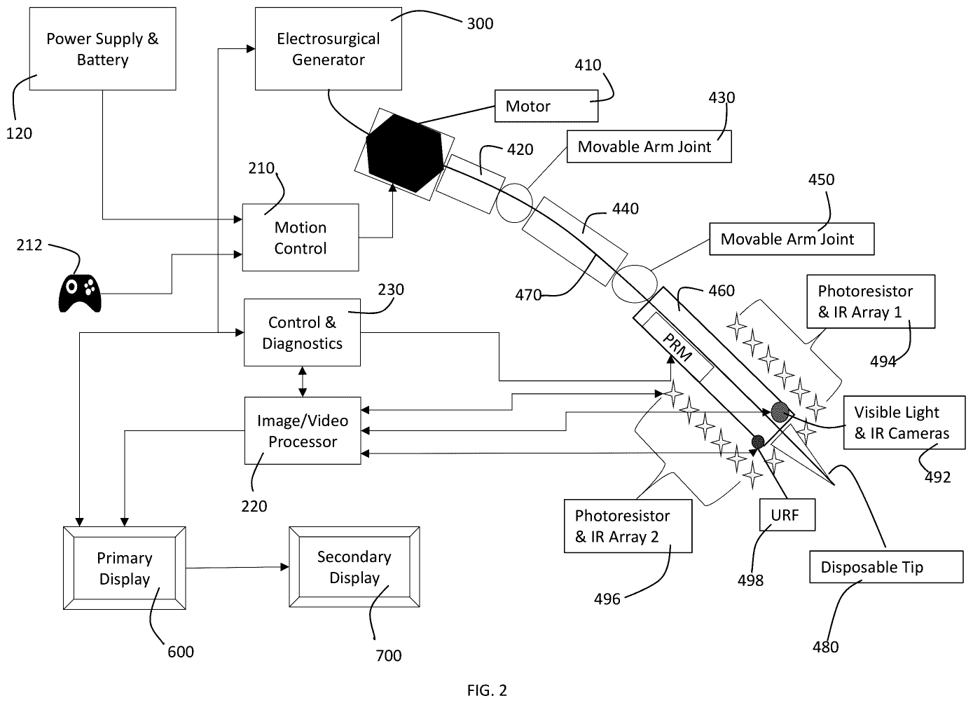

FIG. 2 illustrates interaction between the surgical management system 200 and the robotic arm 400. The robotic arm 400 may have, for example, a motor unit 410, a plurality of link sections 420, 440, 460, a plurality of moveable arm joints 430, 450 and a channel 470 along the length of the arm with an electrode within the channel and connectors for connecting the channel to a source of inert gas and connecting the electrode to electrosurgical generator 300 (the source of electrical energy). Still further, the robotic arm may have a second electrode, for example, a ring electrode, which may be used in procedures such as cold atmospheric plasma procedures. The robotic arm further may have structural means for moving the disposable tip or tool 480, for example, to rotate the tip. An example of a robotic surgical arm that may be used with the present invention is disclosed in PCT Patent Application Serial No. PCT/US2017/053341, which is hereby incorporated by reference in its entirety. The motor 410 may be powered by a battery, from the electrosurgical unit 300, from a wall outlet, or from another power source.

The motion control module 210 and other elements of the surgical management system are powered by a power supply and/or battery 120. The motion control module 210 is connected to an input device 212, which may be, for example, a joystick, keyboard, roller ball, mouse or other input device. The input device 212 may be used by an operator of the system to control movement of the robotic arm 400, functionality of the surgical tool, control of the sensor array 490, and other functionalities of the system 100.

The robotic arm 400 have at or near its distal end a sensor array 490, which comprises, for example, of a plurality of photoresistor arrays 494, 496, visible light and infrared (IR) cameras 492, a URF sensor 498, and other sensors.

The electrosurgical unit 300 preferably is a stand-alone unit(s) having a user interface 310, an energy delivery unit 320 and a gas delivery unit 330. The electrosurgical unit preferably is capable of providing any necessary medium i.e. RF electrosurgery, Cold Atmospheric Plasma, Argon Plasma Coagulation, Hybrid Plasma, etc. For example, a Cold Plasma Generator (CPG) can provide Cold Plasma through tubing that will be fired from a disposable scalpel or other delivery mechanism located at the end closest to the patient. The CPG will receive all instructions from the Surgical Management System (SMS), i.e. when to turn on and off the cold plasma. Preferably the electrosurgical generator has a user interface. While the electrosurgical unit 300 preferably is a stand-alone unit, other embodiments are possible such that the electrosurgical unit 300 comprises and electrosurgical generator and a

The displays 600, 700 are multifaceted and can display power setting, cold plasma status, arm/safe status, number of targets, range to each target, acquisition source, and two crosshairs (one depicting the center of the camera and the other depicting the cold plasma area of coverage). The arm/safe status will provide the surgeon the ability to restrict all cold plasma dispersion until the system is “armed”. The number of targets is determined using “radar-like” device in the sensor array. This device will scan a given area based off the parameters set by programmable signal processor and the use of various photo resistors located throughout the Sensor Array. The range to each target will be either automatic range-which is determined using the 3-D mapping of signal processor and photo resistors in combination with the “radar-like” device—or a ultrasonic range detector (URD) (if the target is in front of the sensor array) and an IR range detector (IRRD) (if the target is located on the sides of the sensor array). The acquisition source is what aligns the camera to the selected target. The surgeon will have two options-select a target from the target array or manual. The target array is built from the positive identifications discovered during each radar sweep and will populate a list within the CPP (Cold Plasma Processor) and will allow the surgeon the select each target on the display. The surgeon can also select “Manual” move the camera and CP (Cold Plasma) tip.

The surgical management system may provide fluorescent image overlay of real-time video on the primary display 600 and/or secondary display 700. Fluorescent imaging from the sensor array 490 may be used by the surgical management system to provide visual servo control of the robotic arm, for example, the cut and/or grasp a tumor. Additionally, using the fluorescent imaging capabilities of the sensor array 490, the surgical management system can provide visual servo control of the robotic arm to treat tumor margins with cold plasma.

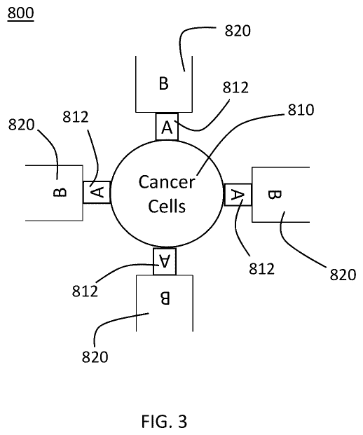

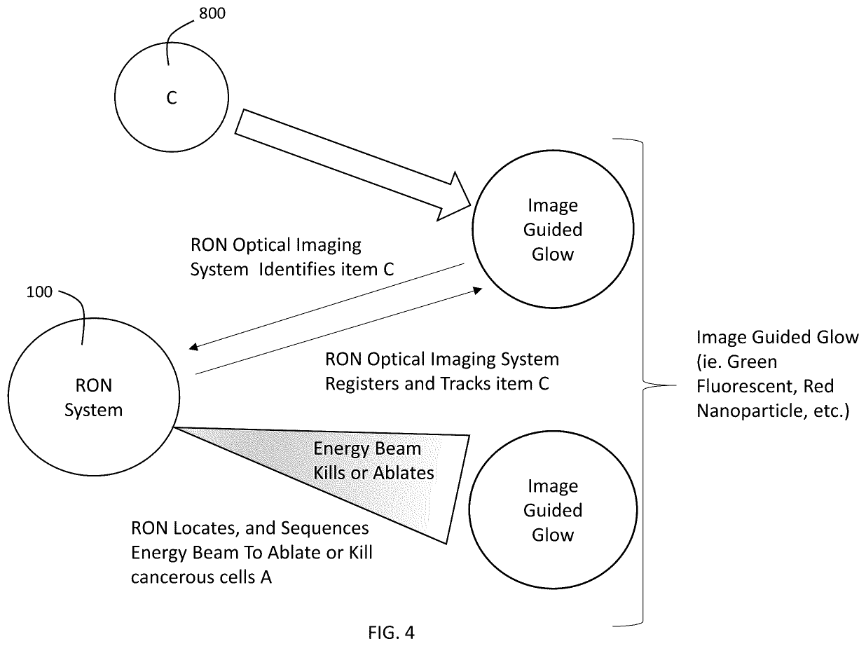

An exemplary method using a robotic navigation system in accordance with the present invention is described with referenced to FIGS. 3-4. As a preliminary step, a resectable portion of the cancerous tissue may be removed from the patient. Such resection may leave cancerous tissue around the margins. Such cancerous tissue in the margins may be treated with the system and method of the present invention. A robotic optical navigation system (“CRON”) of the present invention can be used to locate cancerous tissue around the margins and sequence an energy beam on to the cancerous tissue to ablate or kill that tissue.

As shown in FIG. 3, cancerous cells 810 have over expressed biomarker receptors 812. Through fluorescent imaging methods, an optical smart beacon or die 820 may injected into or applied to the cancerous tissue (and surrounding tissue) such that the dye or smart beacon 820 attaches to the biomarker receptor 812 on the cancerous tissue 810. A variety of such systems such nano-particle guidance, fluorescent protein, or spectral meter may be used with the present invention. In this manner, marked cancerous tissue 800 can be prepared for treatment using the present system.

The sensor array 490 of the robotic optical navigation (RON) system 100 identifies (or locates) an over expressed biomarker receptor A plus an optical smart beacon B complex (marked cancerous tissue 800), the combination of which produces a fluorescent glow C that is sensed by the sensor array 490 and identified by the surgical management system. The robotic optical navigation system then sequences an energy beam—for example, cold atmospheric plasma-onto the cancerous A+B Complex to ablate or kill the tissue.

A broader description of the method is to (1) identify a plurality of locations for treatment; (2) inject a dye that will attach to cancerous tissue to the plurality of locations; (3) sense first target tissue with the sensors in the robotic optical navigation system; (4) verify the first target tissue with the surgical management system; (5) treat the target tissue; (6) sense second target tissue; (7) verify the second target tissue with the surgical management system; and (8) treat the second target tissue. The steps can be repeated for as many target tissues or locations as necessary.

In an alternative embodiment, the system has a channel for delivering a treatment to the cancerous tissue such as with an injection. For example, stimulated media such as is disclosed in U.S. Published Patent Application No. 2017/0183631 could be injected into or applied to the cancerous tissue via the robotic optical navigation system of the present invention. Other types of treatments, such as adaptive cell transfer treatments developed from collecting and using a patient's immune cells to treat cancer could be applied using the robotic optical navigation system of the present invention. See, “CAR T Cells: Engineering Patients' Immune Cells to Treat Their Cancers,” National Cancer Institute (2017).

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A method for performing robotic surgical treatments comprising:
scanning a patient for cancerous tissue in a plurality of regions in said patient;
storing in a memory images of first and second regions of cancerous in said patient;
analyzing cancerous tissue in each of said first and second regions of cancerous tissue to identify a type of cancerous tissue in each of the first and second regions of cancerous tissue;
determining a first specific cold atmospheric plasma dosage and treatment settings for cancerous tissue in said first region of cancerous tissue;
determining a second specific cold atmospheric plasma dosage and treatment settings for cancerous tissue in said second region of cancerous tissue;
programming a robotic surgical system to move to the first region of cancerous tissue, locate cancerous tissue in that region, and apply cold atmospheric plasma of said first specific dosage and treatment settings to the first cancerous tissue, after completion of treatment of the first region move to the second region, locate the cancerous tissue in the second region and apply cold atmospheric plasma to that second cancerous tissue of the second specific dosage and settings.

2. A method for performing robotic surgical treatments according to claim 1 wherein said robotic surgical system locates cancerous tissue in a region by comparing stored images of said region to real-time images of said region.

3. A method for performing robotic surgical treatments according to claim 1, further comprising detecting with a sensor array in said robotic surgical system a distance between an accessory attached to said robotic surgical system and target tissue.

4. A method for performing robotic surgical treatments according to claim 3, further comprising controlling movement of said accessory with said robotic surgical system to maintain a constant distance from said accessory to target tissue based upon results of said detecting and a 3-D map of an area of target tissue.
